(19) United States
(12) Patent Application Publication
(10) Pub. No.: US 2005/0191276 A1
(43) Pub. Date: Sep. 1, 2005

(54) Treatment of inflammatory bowel disease through induction of indoleamine 2.3-dioxygenase

(76) Inventors: Gregory Gurtner, Ridgefield, CT (US); Nancy Gurtner, Ridgefield, CT (US); William F. Stenson, Ladue, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/997,147

(22) Filed: Nov. 24, 2004

Related U.S. Application Data
(60) Provisional application No. 60/531,587, filed on Dec. 19, 2003.
(60) Provisional application No. 60/524,753, filed on Nov. 25, 2003.

(51) Int. Cl. A61K 38/21; A61K 31/739

(52) U.S. Cl. 424/85.6; 514/54

(57) ABSTRACT

Methods and compositions for treating inflammatory bowel disease based upon increasing activity of indoleamine 2,3-dioxygenase in antigen presenting cells are disclosed.

### GOVERNMENT INTERESTS

This work was supported at least in part with funds from the federal government under U.S.P.H.S. Grant P30 DK52574 awarded by the National Institutes of Health. The U.S. Government may have certain rights in the invention.

### FIELD

This application relates generally Inflammatory Bowel Diseases and, more particularly, to methods and compositions for treating Inflammatory Bowel Diseases.

### BACKGROUND

Inflammatory bowel diseases including Crohn's disease and ulcerative colitis, are chronic inflammatory disorders of the gastrointestinal tract resulting from upregulation of mucosal immune system. Current treatment approaches involve the use of antiinflammatory agents, aminosalicylates and corticosteroids. (for review, see Hibi et al., J. Gartroenterol 38 Suppl 15:36-42, 2003). Nevertheless, these therapies do not successfully treat all patients, and in patients in whom the therapies are effective, unpleasant side effects are often seen (Sawada, Dis Colon Rectum 46(10 Suppl):S66-S77, 2003). Thus, there remains a need for new therapeutic approaches.

### SUMMARY

Accordingly, the present inventors have succeeded in discovering that increased expression of indoleamine 2,3-dioxygenase in antigen presenting cells of the gastrointestinal tract produces a downregulation of the proliferative response of Th1 helper-T cells during inflammation. As a result, substances that increase concentration or activity of indoleamine 2,3-dioxygenase in antigen presenting cells of the gastrointestinal tract, decrease the inflammatory response and in patients having inflammatory bowel disease. This provides a new treatment approach for the disease.

Thus, in various embodiments, the present invention can involve a method for treating a patient having inflammatory bowel disease. The method can comprise administering to the patient an anti-inflammatory amount of an inducer of indoleamine 2,3-dioxygenase in antigen presenting cells of the patient's gastrointestinal tract. By the term “inducer” or “inducers” reference is made to compounds that increases activity of indoleamine 2,3-dioxygenase in the cell such as by increasing the amount of the enzyme or by increasing the substrate turnover rate. Such increase in enzyme activity reduces inflammation in the gastrointestinal tract and thereby provides a new approach for treating inflammatory bowel disease.

The present invention, in various embodiments, can also involve a method for downregulating a T helper 1 cell proliferation response in inflammation. The T helper 1 cell can be within the gastrointestinal tract in a mammalian patient having inflammatory bowel disease. The method can comprise administering to the patient a pharmaceutical composition comprising an inducer of indoleamine 2,3-dioxygenase in antigen presenting cells. The inducer can increase expression of the enzyme. This results in the downregulation of T helper 1 cell proliferation response in the inflammation of inflammatory bowel disease.

In various other embodiments, the present invention is also directed to a packaged pharmaceutical. The packaged pharmaceutical can comprise an anti-inflammatory amount of an inducer of indoleamine 2,3-dioxygenase in antigen presenting cells of a patient having inflammatory bowel disease. The substance is in a pharmaceutically acceptable formulation. The package pharmaceutical can also include instructions for using said substance for treating inflammatory bowel disease in a patient.

In various of the embodiments of the present invention the inflammatory bowel disease can be ulcerative colitis or Crohn's disease and the substance administered can increase expression of indoleamine 2,3-dioxygenase in the antigen presenting cells. Non-limiting examples of inducers of indoleamine 2,3-dioxygenase include a bacterial lipopolysaccharide, a cytokine such as interferon gamma or a cytotoxic T lymphocyte-associated antigen. The cytokine or cytotoxic T lymphocyte-associated antigen 4 can be in the form of a fusion polypeptide or a pegylated polypeptide.

### BRIEF DESCRIPTION OF THE DRAWINGS

FIG. 1 illustrates increased Indoleamine 2,3-dioxygenase expression in the distal colon in Trinitrobenzene Sulfonic Acid colitis as demonstrated by A) Real-Time PCR and B) Western blotting.

FIG. 2 illustrates increased Indoleamine 2,3-dioxygenase expression in lamina propria mononuclear cells in Trinitrobenzene Sulfonic Acid-treated colons and decreased quinolinic acid expression in Trinitrobenzene Sulfonic Acid-treated colons treated with 1-methyl tryptophan.

FIG. 3 illustrates worsening colitis and increased mortality in Trinitrobenzene Sulfonic Acid colitis with Indoleamine 2,3-dioxygenase inhibition.

FIG. 4 illustrates that Indoleamine 2,3-dioxygenase inhibition leads to toxic colonic dilation in the setting of Trinitrobenzene Sulfonic Acid colitis.

FIG. 5 illustrates the increase in cytokines expressed in colons of mice treated with Trinitrobenzene Sulfonic Acid and placebo or 1-methyl tryptophan using Real-Time PCR to quantify mRNA levels.

FIG. 6 illustrates Indoleamine 2,3-dioxygenase expression in lamina propria mononuclear cell subpopulations isolated by fractionation using magnetic selection columns.

FIG. 7 illustrates decreased Indoleamine 2,3-dioxygenase expression and increased inflammation in STAT-1 knockout mice exposed to Trinitrobenzene Sulfonic Acid.

FIG. 8 illustrates decreased Indoleamine 2,3-dioxygenase expression in IFN-γ Receptor and STAT-1 knockout mice.

FIG. 9 illustrates that inhibition of Indoleamine 2,3-dioxygenase by 1-methyl tryptophan leads to increased lymphocyte proliferation in Trinitrobenzene Sulfonic Acid colitis as detemined by BrdU immunohistochemistry.

FIG. 10 illustrates that LPS or CTLA-4-Ig administration induces Indoleamine 2,3-dioxygenase expression in lamina propria mononuclear cells and LPS downregulates the inflammatory response to Trinitrobenzene Sulfonic Acid.

### DETAILED DESCRIPTION

Thus, the inflammation response in inflammatory bowel disease can be treated by increasing expression of indoleamine 2,3-dioxygenase in antigen presenting cells of the gastrointestinal tract which results in the downregulation of the proliferative response of Th1 helper-T cells. This is also the subject of copending U.S. Patent Application No. 60/524,753 filed Nov. 25, 2003, which is incorporated herein by reference in its entirety.

Diagnosis of inflammatory bowel disease is generally based upon evaluation of a patient's clinical, radiographic, endoscopic, and histopathologic features. (for review see Papadakis et al., Gastrointest. Endosc. Clin. N. Am. 12:433-449, 2002; Chutkan et al., Gastrointest. Clin. N. Am. 12:463-483, 2002; Fishman, Can. J. Gastroenterol. 15:627-628, 2001). The inventors herein have shown that expression of indoleamine 2,3-dioxygenase is increased in an animal model of colitis predictive of inflammatory bowel disease in humans (for review see Neurath et al., Int. Rev. Immunol. 19:51-62, 2000), and that inhibition of the enzyme leads to a worsening of colitis symptoms in the animal model, including increased mortality. Hence, it is believed that this increase in expression can in certain instances be of benefit, at least in part, in the diagnosis of inflammatory bowel disease, i.e. in distinguishing the disease from other diseases.

Furthermore, increasing the concentration of the indoleamine 2,3-dioxygenase by increasing its expression has a beneficial effect in reducing inflammation in inflammatory bowel disease in humans thereby providing a new treatment approach. As used herein, the term “treatment” is intended to include at least a partial relief of symptoms of the disease up to and including complete abrogation of the disease. Treatment also includes preventing the development of the disease by administration of inducer compounds prior to the appearance of clinical symptoms or very early in the course of the disease before significant clinical symptoms and/or pathological changes have occurred.

A number of substances are known to increase the activity of indoleamine 2,3-dioxygenase in cells. Substances having this activity are effective in treating inflammatory bowel disease and within the scope of the present invention. One example of such substances is cytolytic T lymphocyte-associated antigen 4. The water soluble fusion protein of this substance, i.e. cytolytic T lymphocyte associated antigen 4-immunoglobulin has been shown to induce indoleamine 2,3 dioxygenase in dendritic cells (Mellor et al, J. Immunol. 171:1652-1655, 2003; Grohmann et al, Nat. Immunol. 3:1097-1101, 2002). Thus, both cytolytic T lymphocyte-associated antigen 4 immunoglobulin and cytolytic T lymphocyte-associated antigen 4 can increase expression of the enzyme and diminish inflammation in inflammatory bowel disease (for commercial availability, see BD Biosciences Pharmingen, San Diego, Calif.).

Interferon-gamma has also been shown to increase indoleamine 2,3 dioxygenase levels by increasing expression of the enzyme (Kudo et al., Mol. Hum. Reprod, 6:369-374, 2000). Recombinant Human Interferon-gamma in lyophilized form is very water soluble and it is readily available commercially (see for example, BD Biosciences Pharmingen, San Diego, Calif.).

Bacterial lipopolysaccharide has been shown to be an inducer of indoleamine 2,3 dioxygenase (see Fujigaki et al., Eur. J. Immunol. 31:2313-1318, 2001; Lestage et al, Brain Behav. Immun. 16:596-601, 2002; Hwu et al. J. Immunol. 164:3596-3599, 2000). The inventors herein have also have shown bacterial lipopolysaccharide to be a weak inducer of indoleamine 2,3-dioxygenase. The lipopolysaccharide can be obtained from Enterobacteriaceae such as E. Coli or Salmonella species (for commercial availability, see Sigma-Aldrich, St. Louis, Mo.).

These and other substances can be used in the present invention. In certain aspects of the present invention, the substances can be pegylated, i.e. stably linked to polyethylene glycol, to obtain desirable properties of solubility, stability, half-life and other pharmaceutically advantageous properties. (see for example, Francis et al., Int. J. Hematol. 68:1-18, 1998).

The beneficial effect of substances that induce indoleamine 2,3-dioxygenase can be a diminished inflammation in inflammatory bowel disease, but not necessarily a full remediation of the disease. Thus, it is envisaged by the inventors herein that substances that induce indoleamine 2,3-dioxygenase can be used in treatment regimens that include other agents such as, for example, 5-aminosalicylates, corticosteroids and azathioprine. Any one or more of such treatments can be combined with the substances that induce indoleamine 2,3-dioxygenase of the present invention.

The substances of the present invention which increase activity of indoleamine 2,3-dioxygenase can be in pharmaceutically acceptable formulations or preparations. Such formulations are suitable for therapeutic use in patients following administration by any suitable route including parenteral and oral routes of administration such as for example, intraperitoneal, intravenous, subcutaneous, intramuscular, intranasal transdermal, oral and the like. Administration can be either rapid as by injection or over a period of time as by slow infusion or administration of controlled release formulation.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The substances can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Such excipients are those substances usually and customarily employed to formulate dosages for parenteral administration in either unit dosage or multi-dose form or for continuous or periodic infusion.

Dose administration can be repeated depending upon the pharmacokinetic parameters of the dosage formulation and the route of administration used.

It is also contemplated that certain formulations containing the substances of the present invention are to be administered orally. Such formulations can be encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose can be calculated according to the approximate body weight or body surface area of the patient or the volume of body space to be occupied. Another factor in considering the appropriate dose can be the particular route of administration selected. Further refinement of the calculations necessary to determine the appropriate dosage for treatment is routinely made by those of ordinary skill in the art. Such calculations can be made without undue experimentation by one skilled in the art. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In various embodiments, the present invention includes packaged pharmaceuticals. The packaged pharmaceutical can comprise an anti-inflammatory amount of an inducer that increases activity of indoleamine 2,3-dioxygenase in antigen presenting cells of a patient having inflammatory bowel disease. The substance can be in a pharmaceutically acceptable formulation. The package pharmaceutical can also include instructions for using said substance for treating inflammatory bowel disease in a patient. Such instructions can be in the form of a package insert, a pamphlet or a computer-readable form such as floppy disc, compact disc and the like.

The invention can be further understood by reference to the examples which

### EXAMPLE 1

This example illustrates the cellular distribution of indoleamine 2,3-dioxygenase protein in the normal colon using immunohistochemistry.

We determined the presence of indoleamine 2,3-dioxygenase protein in lamina propria mononuclear cells and vascular endothelial cells in the colon as follows. The colons of SJL/J mice were removed and fixed in 10% formalin overnight and then transferred to 70% ethanol. After embedding in paraffin, 4 μm serial sections were prepared. Endogenous peroxidase was quenched for 30 minutes in 1% hydrogen peroxide/PBS. The sections were then treated with a solution of Nuclear Decloaker (Biocare Medical, Walnut Creek, Calif.) in a pressure cooker at 15 PSI for 3 minute and then with Avidin/Biotin blocking (Vector Lab., Burlingame, Calif.) for 20 minutes each. The sections were treated with Protein Block (Dako, Carpenteria, Calif.) for 10 minutes, and then incubated with indoleamine 2,3-dioxygenase primary antibody 1:100 at 30° C. for 1 hr.

To make the antibody, mouse indoleamine 2,3-dioxygenase cDNA was first obtained from mouse colon total RNA by reverse transcription and PCR amplification. The cDNA was cloned in the bacterial expression vector, pET28a (Novagen, Madison Wis.), and recombinant indoleamine 2,3-dioxygenase protein was expressed in Novablue (DE3) cells (Novagen). Purified protein was isolated using His-binding affinity columns and used to immunize rabbits (Cocalico Biologicals, Reamstown, Pa.). Mouse indoleamine 2,3-dioxygenase antibody was purified by Protein-A Sepharose column chromatography separation of rabbit serum, followed by affinity purification with recombinant mouse indoleamine 2,3-dioxygenase. The secondary antibody, goat anti-rabbit biotinylated IgG (NEN Life Science, Boston, Mass.), was applied for 30 minutes 1:1000 at 30° C. The sections were incubated with SA-HRP (P0397, Dako, Carpenteria, Calif.) 1:1000 for 30 minutes at 30° C. and rinsed. DAB (D9015, Sigma, St. Louis, Mo.) was applied until staining was evident microscopically. The tissue sections were counterstained with hematoxylin.

Immunohistochemistry for detecting quinolinic acid protein was performed by perfusing mice with transcardial carbodiimide to form amide bonds between the carboxyl groups on quinolinic acid and the primary amines on tissue proteins. Tissues were removed and fixed in Bouin's solution. Anti-quinolinic acid antibodies were obtained by raising polyclonal antiserum, as described for indoleamine 2,3-dioxygenase but against quinolinic acid (quinolinic acid) conjugated to bovine thyroglobulin (as previously described by Moffett J R, Cell Tissue Res 278:461-469, 1994). Anti-quinolinic acid antibodies were utilized for immunohistochemistry using the Vectastain Elite Kit (Vector Lab., Burlingame, Calif.) per manufacturer's instructions in conjunction with Avidin/Biotin blocking.

Immunostaining of tissues revealed the presence of indoleamine 2,3-dioxygenase protein in the endothelium of arteries in the lamina propria and the mesentery. Endothelial cells in veins did not express indoleamine 2,3-dioxygenase. Indoleamine 2,3-dioxygenase protein in lamina propria mononuclear cells was not detected using Bouin's-fixed or formalin-fixed sections; however, immunostaining of unfixed frozen sections revealed indoleamine 2,3-dioxygenase expression in a population of lamina propria cells with a morphology consistent with fibroblasts or dendritic cells. Cells with the same morphology and localization stain strongly for quinolinic acid, a product of tryptophan metabolism through indoleamine 2,3-dioxygenase.

These studies established that indoleamine 2,3-dioxygenase is expressed in the colon in arterial endothelial cells and a population of lamina propria mononuclear cells with the morphology of fibroblasts and/or dendritic cells. Not only was indoleamine 2,3-dioxygenase expressed in the normal colon lamina propria, but the protein itself was active as demonstrated by the presence of quinolinic acid, a metabolite of tryptophan through the kynurenine pathway, seen in cells with the same morphology as those expressing indoleamine 2,3-dioxygenase.

### EXAMPLE 2

This example illustrates the increase in indoleamine 2,3-dioxygenase in cells of the colon after treatment with Trinitrobenzene Sulfonic Acid.

Six week old female SJL/J mice weighing approximately 20 grams, which were maintained at a controlled temperature and light/dark cycle in a pathogen free facility, were anesthesized with an intraperitoneal injection of a 10% Ketamine/Xylazine mixture. Colitis was induced by intrarectal administration of 0.5 mg of Trinitrobenzene Sulfonic Acid in 35% ethanol via a flexible 3.5 Fr catheter inserted 4 cm proximal to the anus. Inhibition of indoleamine 2,3-dioxygenase was achieved by surgical insertion of slow release 1-methyl tryptophan pellets under the dorsal skin at the time of Trinitrobenzene Sulfonic Acid administration, whereas control mice received placebo pellets. The pellets released 1-methyl tryptophan at a constant rate of 0.9 mg/hr for a period of 10 days. Mice were sacrificed 4 days after treatment for determination of indoleamine 2,3-dioxygenase protein and mRNA levels.

Western blotting (Bio-Rad, Hercules, Calif.) for determining indoleamine 2,3-dioxygenase protein amount was performed on whole colon lysates obtained from the distal colons of both control mice and mice treated with Trinitrobenzene Sulfonic Acid. For Lamina Propria Mononuclear cells, 1×10⁶ cells were concentrated and loaded per lane. The samples were denatured and separated on an 8% Sodium Dodecyl Sulphate-Polyacrylamide (SDS-PAGE) gel. After electrophoresis, the separated proteins were then transferred to an Immobilon-P Transfer Membrane (Milipore, Bedford, Mass.). Indoleamine 2,3-dioxygenase protein was detected with mouse indoleamine 2,3-dioxygenase primary antibody described in Example 1 above using ECL (Amersham). To make this antibody, mouse indoleamine 2,3-dioxygenase cDNA was first obtained from mouse colon total RNA by reverse transcription and PCR amplification. The cDNA was cloned in the bacterial expression vector, pET28a (Novagen. Madison Wis.), and recombinant indoleamine 2,3-dioxygenase protein was expressed in Novablue (DE3) cells (Novagen). Purified protein was isolated using His-binding affinity columns and used to immunize rabbits (Cocalico Biologicals, Reamstown, Pa.). Anti-mouse indoleamine 2,3-dioxygenase antibody was purified by Protein-A Sepharose column chromatography separation of rabbit serum, followed by affinity purification with recombinant mouse indoleamine 2,3-dioxygenase. The secondary antibodies were donkey anti-rabbit linked to horseradish peroxidase. After probing for indoleamine 2,3-dioxygenase protein, the membranes were stripped and reprobed for β-actin, which was used in addition to the protein assay to ensure equal protein loading.

Real-Time PCR for determining indoleamine 2,3-dioxygenase mRNA amount was performed using primers designed for the mouse indoleamine 2,3-dioxygenase gene, as well as various cytokines using Primer Express Software. Primers were synthesized by the Protein and Nucleic Acid Chemistry Lab at Washington University. Total RNA was isolated from homogenized distal SJL/J mouse colon using Trizol per manufacturer's directions (Invitrogen, Carlsbad, Calif.). Reverse transcription was performed using random primers, dNTPs, and Superscript II (Invitrogen). Mouse c-DNA was then used to perform real-time PCR using SYBR Green PCR Master Mix (Applied Biosystems Foster City, Calif.) as the detection system in the i-Cycler (Bio-Rad) or the ABI PRISM 7000 Sequence Detection System (Applied Biosystems). The PCR products were validated by melt analysis.

Immunoflourescence for detecting indoleamine 2,3-dioxygenase protein was performed on fresh-frozen colon sections of control and Trinitrobenzene Sulfonic Acid-treated mice, which were prepared by freezing in TISSUE-TEK O.C.T Compound (Miles, Elkhart Ind.). Sections were cut at 6 microns and washed in 95% ethanol. They were then blocked in TNB solution for 30 minutes. The primary anti-indoleamine 2,3-dioxygenase used was as described in Example 1 above. The secondary antibody was an anti-rabbit IgG conjugated to Rhodamine Red (Jackson Immuno-research, West grove, Pa.). DAPI (DAKO Corperation Carpinteria, Calif.) was then used for nuclear counterstaining.

Immunohistochemistry for detecting quinolinic acid protein was performed as described above in Example 1.

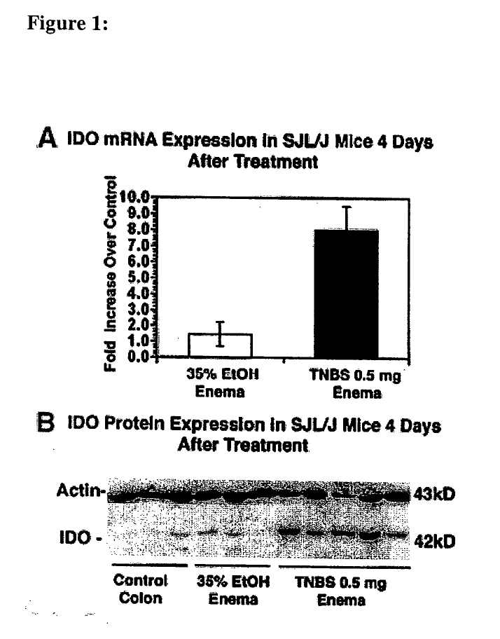

We determined whether the colitis induced in mice after treatment with Trinitrobenzene Sulfonic Acid is associated with increased amounts of indoleamine 2,3-dioxygenase mRNA and protein. Real-time PCR analysis of lysates from the distal colons of mice 4 days after treatment with Trinitrobenzene Sulfonic Acid showed a significant 8-fold increase in indoleamine 2,3dioxygenase mRNA amounts when compared with untreated control mice (FIG. 1A). In mice receiving Trinitrobenzene Sulfonic Acid plus 1-methyl tryptophan, indoleamine 2,3-dioxygenase mRNA amounts were similar to that in mice receiving Trinitrobenzene Sulfonic Acid plus placebo (not shown). Administration of a 35% ethanol enema without Trinitrobenzene Sulfonic Acid had no significant effect on indoleamine 2,3-dioxygenase mRNA levels compared with control animals.

Induction of indoleamine 2,3-dioxygenase protein by Trinitrobenzene Sulfonic Acid administration was demonstrated by Western blotting techniques. A 42-kilodalton band representing indoleamine 2,3-dioxygenase was barely detectable in distal colon lysates from untreated mice. Treatment with a 35% ethanol enema alone did not induce indoleamine 2,3-dioxygenase protein amount. But, four days after Trinitrobenzene Sulfonic Acid administration, there was a marked induction of indoleamine 2,3-dioxygenase in the distal colon (FIG. 1B). There was no difference in colonic indoleamine 2,3-dioxygenase protein amount between mice receiving Trinitrobenzene Sulfonic Acid plus placebo and those receiving Trinitrobenzene Sulfonic Acid plus 1-methyl tryptophan.

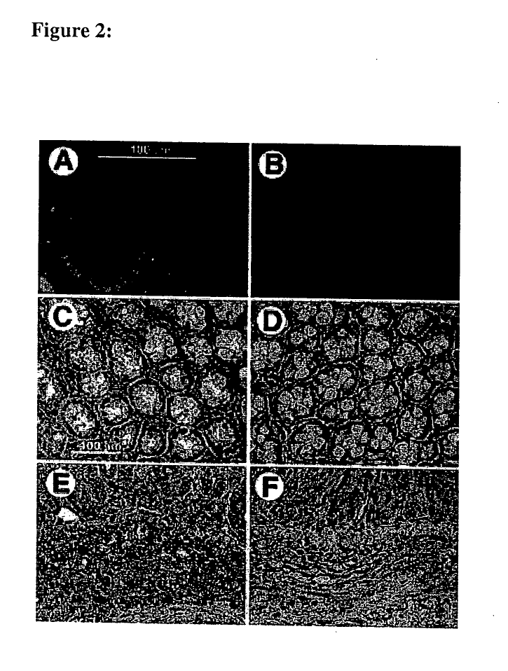

We also determined whether, in the setting of Trinitrobenzene Sulfonic Acid colitis, indoleamine 2,3-dioxygenase protein amount and activity are increased in colonic lamina propria mononuclear cells. Frozen sections of mouse colon demonstrated low baseline indoleamine 2,3-dioxygenase staining in the cytoplasm of mononuclear cells in the lamina propria surrounding the colonic crypts (FIG. 2A). In the setting of Trinitrobenzene Sulfonic Acid colitis, there was an increase in the staining intensity within individual cells as well as an increase in the number of staining cells (FIG. 2B). Lamina propria mononuclear cells with the same morphology and localization stained strongly for quinolinic acid, the product of tryptophan metabolism through indoleamine 2,3-dioxygenase (FIGS. 2C and D). There was decreased quinolinic acid staining in the colons of mice treated with 1-methyl tryptophan versus placebo both in the presence and absence of Trinitrobenzene Sulfonic Acid colitis (FIGS. 2C and F). This suggested diminished indoleamine 2,3-dioxygenase activity in the presence of this indoleamine 2,3-dioxygenase inhibitor.

These studies established that indoleamine 2,3-dioxygenase protein and mRNA amount are increased in the colon in the setting of Trinitrobenzene Sulfonic Acid colitis, a T cell Helper 1-mediated model. These studies further showed indoleamine 2,3-dioxygenase expression at baseline in lamina propria mononuclear cells in the colon, with a marked increase in amounts in these cells in Trinitrobenzene Sulfonic Acid colitis. Moreover, inhibition of indoleamine 2,3-dioxygenase with 1-methyl tryptophan resulted in decreased amounts of quinolinic acid in the colons of both untreated and Trinitrobenzene Sulfonic Acid-treated mice. Quinolinic acid is a catabolite of tryptophan through the kynurenine pathway; decreased quinolinic acid levels in the 1-methyl tryptophan-treated mice demonstrate that the drug achieved its predicted pharmacologic effect of inhibiting indoleamine 2,3-dioxygenase in the colon. 1-methyl tryptophan is a known competitive inhibitor of indoleamine 2,3-dioxygenase.

### EXAMPLE 3

This example illustrates the increase in mortality in Trinitrobenzene Sulfonic Acid colitis with indoleamine 2,3-dioxygenase inhibition.

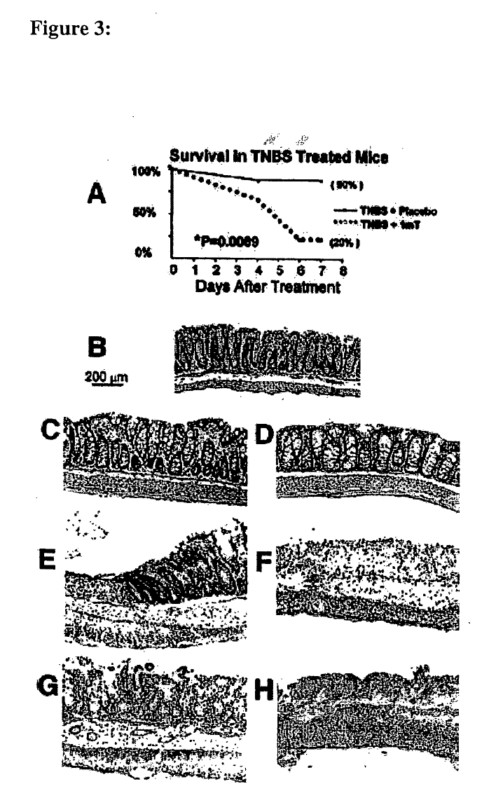

Mice received Trinitrobenzene Sulfonic Acid per rectum in addition to a subcutaneous pellet containing either placebo or 1-methyl tryptophan. Of the 10 mice that received Trinitrobenzene Sulfonic Acid plus 1-methyl tryptophan, 3 died within 4 days of Trinitrobenzene Sulfonic Acid administration. Of the remaining 7 mice, 5 developed tensely dilated stool-filled colons prior to death on days 4 to 6 after Trinitrobenzene Sulfonic Acid administration. Of the mice that received Trinitrobenzene Sulfonic Acid plus placebo, only 1 died, and none of the others developed significant dilation or stool retention. The survival rate was 100% in mice receiving a 35% ethanol enema along with either placebo or 1-methyl tryptophan (not shown). For mice receiving Trinitrobenzene Sulfonic Acid plus placebo, there was a 90% survival at 7 days after Trinitrobenzene Sulfonic Acid administration (FIG. 3A). In contrast, only 20% of the mice receiving Trinitrobenzene Sulfonic Acid plus 1-methyl tryptophan were still alive 7 days after Trinitrobenzene Sulfonic Acid treatment (FIG. 3A). Survival data were assessed using a χ² test. These studies demonstrated that inhibition of indoleamine 2,3-dioxygenase affects the course of the Th1-mediated Trinitrobenzene Sulfonic Acid model of colitis resulting in increased mortality.

### EXAMPLE 4

This example illustrates the effect of indoleamine 2,3-dioxygenase inhibition on gross morphology and histology in Trinitrobenzene Sulfonic Acid colitis.

The colon was removed from its mesentery to the pelvic brim by blunt dissection and the serosal surface examined under a dissecting microscope. The colon was then opened longitudinally along the mesenteric attachment and then pinned flat so that the mucosal surface could be examined. A modification of a scoring scale (Colon et al., Cytokine 15: 220-226, 2001) was used to assess the degree of macroscopic inflammation in the distal colon (1: normal mucosa; 2: edema and hyperemia; 3: small ulcers with mild intraperitoneal adhesions; 4: large ulcers [>7 mm]±extensive intraperitoneal adhesions; 5: megacolon, perforation, and necrosis).

The pinned out colon was then fixed in 10% formalin overnight and then transferred to 70% ethanol. After embedding in paraffin, 4 μm serial sections were prepared and stained with hematoxylin and eosin for histologic grading. A modification of the scoring scale of Fuss et al., J. Immunol. 168: 900-908, 2002 was used to assess the microscopic degree of inflammation on longitudinal sections of the colon (1: no evidence of inflammation; 2: low level of lymphocyte infiltration with infiltration seen in a <10% high-power field (hpf), no structural changes observed; 3: moderate lymphocyte infiltration with infiltration seen in 10%-25% hpf, crypt elongation, bowel wall thickening, which does not extend beyond mucosal layer, no evidence of ulceration; 4: high level of lymphocyte infiltration with infiltration seen in 25%-50% hpf, high vascular density, thickening of bowel wall, which extends beyond mucosal layer; 5: marked degree of lymphocyte infiltration with infiltration seen in >50% hpf, high vascular density, crypt elongation with distortion, transmural bowel wall-thickening with ulceration; 6: Complete loss of mucosal architecture (crypts) with ulceration covering >1 low-power field and loss of mucosal vasculature; 7: coagulation necrosis of at least the mucosal layer).

The histologic appearance of Trinitrobenzene Sulfonic Acid colitis was assessed in animals receiving 1-methyl tryptophan or placebo at 3 days, 4 days, and 6 days after Trinitrobenzene Sulfonic Acid administration. On day 3 after Trinitrobenzene Sulfonic Acid administration, there was no significant histologic differences between the 2 groups (Table 1); however, after day 3, the histology in the Trinitrobenzene Sulfonic Acid plus placebo group began to improve, whereas that in the Trinitrobenzene Sulfonic Acid plus 1-methyl tryptophan group became progressively worse. The histologic scores in the group receiving Trinitrobenzene Sulfonic Acid plus 1-methyl tryptophan were significantly higher at day 4 and day 6. There was significant indoleamine 2,3-dioxygenase protein induction in colon lysates starting at around day 3 (not shown). Morphologic and histologic data were assessed using a Student t test.

Mice receiving 35% ethanol enemas demonstrated variable amounts of mucosal injury during the first 2 to 3 days after administration. At day 4, animals receiving a 35% ethanol enema and either placebo or 1-methyl tryptophan to inhibit indoleamine 2,3-dioxygenase demonstrated no ulceration or inflammatory infiltrate (FIG. 3C-D, respectively). There was no significant histologic difference between the 2 ethanol-treated groups and control animals (FIG. 3B) besides occasional goblet cell hyperplasia.

At day 4, animals receiving Trinitrobenzene Sulfonic Acid plus placebo developed areas of focal ulceration associated with transmural infiltration of inflammatory cells and thickening of the colonic wall, in particular in the muscularis (FIG. 3E). The affected area was limited to the region of the distal colon that had likely come into direct contact with Trinitrobenzene Sulfonic Acid. Except for some areas of focal ulceration, there was overall preservation of crypt architecture. By day 6, there was some improvement with fewer areas of focal ulceration (FIG. 3F).

At day 4 in the distal colons of mice receiving Trinitrobenzene Sulfonic Acid plus 1-methyl tryptophan, there was a loss of mucosal architecture and an increased transmural inflammatory infiltrate compared with the colons of mice receiving Trinitrobenzene Sulfonic Acid plus placebo (FIG. 3G). There was more uniform loss of epithelium with extensive circumferential ulceration. There was also a paucity of vessels within the lamina propria. By day 6, this region appears grossly necrotic with a lack of vascularity (FIG. 3H). There was progressive thinning of the colonic wall with persistence of an inflammatory infiltrate. The boundaries between the muscularis, submucosa, and mucosa became less obvious. In severely ill animals, there was complete loss of tissue architecture with evidence of perforation.

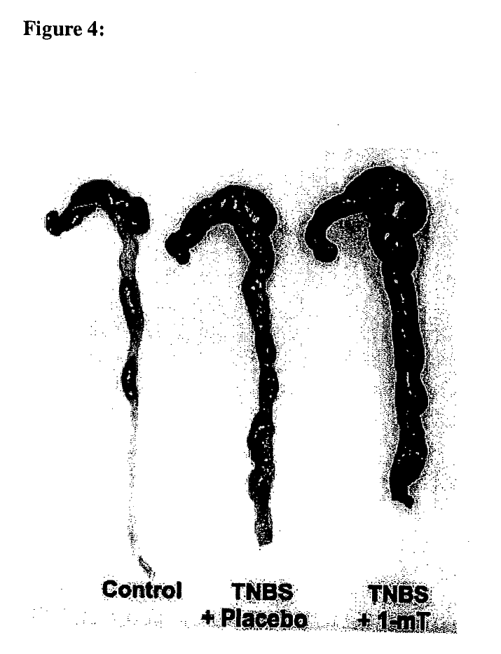

With respect to morphologic appearance of Trinitrobenzene Sulfonic Acid colitis, by day 4 after Trinitrobenzene Sulfonic Acid administration, there were significant gross morphologic differences between the colons of mice treated with Trinitrobenzene Sulfonic Acid plus 1-methyl tryptophan (to inhibit indoleamine 2,3-dioxygenase) and the colons of those treated with Trinitrobenzene Sulfonic Acid plus placebo (P=0.008, Table 1). At this time, there was gross evidence of inflammation and edema in the distal colons of both groups of animals (FIG. 4). However, the colons from the 1-methyl tryptophan-treated mice were significantly more indurated, dilated, and packed with solid stool. Similar to the histology described previously, there were no gross colonic morphologic differences between untreated control mice and mice receiving an ethanol enema with either 1-methyl tryptophan or placebo.

These studies demonstrated that administration of 1-methyl tryptophan had no effect on colonic morphology in untreated mice or in mice receiving 35% ethanol enemas in the absence of Trinitrobenzene Sulfonic Acid. Thus, pharmacologic inhibition of indoleamine 2,3-dioxygenase did not activate the mucosal immune system in the colon either in the absence of injury or in the presence of mild transient colonic injury as is seen with 35% ethanol enemas. However, inhibition of indoleamine 2,3-dioxygenase in mice with Trinitrobenzene Sulfonic Acid colitis resulted in a marked worsening of mucosal histology and increased mortality. The Trinitrobenzene Sulfonic Acid colitis model is a delayed type hypersensitivity response directed against TNP haptenated neoantigens, resulting in Th1 cell activation and, therefore, increased IFN-γ production. Histology in Trinitrobenzene Sulfonic Acid-treated mice receiving either placebo or 1-methyl tryptophan was similar during the first 3 days. Beginning at day 4, however, the histology in the Trinitrobenzene Sulfonic Acid-treated mice receiving placebo began to improve while that in the Trinitrobenzene Sulfonic Acid-treated mice receiving 1-methyl tryptophan continued to worsen. The timing of these events coincided with the appearance of increased indoleamine 2,3-dioxygenase expression typically seen by 3 days after Trinitrobenzene Sulfonic Acid administration. This was further evidence that indoleamine 2,3-dioxygenase was acting to antagonize the Th1 response to this potent immunologic stimulus.

### EXAMPLE 5

This example illustrates the increase in proinflammatory cytokine mRNA amounts within the colons of mice treated with Trinitrobenzene Sulfonic Acid and placebo or 1-mT using Real-Time PCR.

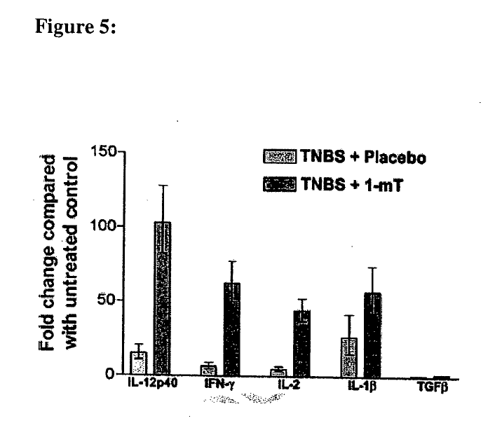

Primers were designed for the various cytokines using Primer Express Software and Real-Time PCR was performed as described in Example 1 above. Real-Time PCR was used to quantify mRNA expression of cytokines in colon lysates from mice treated with either placebo or 1-mT 4 days after Trinitrobenzene Sulfonic Acid administration. There was significant induction over baseline of the proinflammatory cytokines IL-12, IFN-γ, interleukin (IL)-2, and IL-1 in both Trinitrobenzene Sulfonic Acid-treated groups (FIG. 5). There was significantly higher expression by orders of magnitude of IL-12, IFN-γ, and IL-2 in mice receiving Trinitrobenzene Sulfonic Acid plus 1-mT-treated mice versus mice receiving Trinitrobenzene Sulfonic Acid and placebo-treated mice. IL-12 and IFN-γ expression increased 20-fold and 8-fold, respectively, in the placebo-treated group and 120-fold and 75-fold, respectively, in the 1-mT-treated group. In contrast, the expression of the anti-inflammatory cytokine TGF-β was about half of control values in the Trinitrobenzene Sulfonic Acid- and placebo-treated group and 1.5 times control values in the Trinitrobenzene Sulfonic Acid- and 1-mTtreated group.

These studies established the increased levels of proinflammatory cytokines in the colons of mice receiving 1-mT in addition to Trinitrobenzene Sulfonic Acid, which may explain the worsening histology and increased mortality in these mice. IL-12, IFN-γ, IL-2, and IL-1 mRNA levels were all markedly increased in the animals receiving Trinitrobenzene Sulfonic Acid plus 1-mT as compared with those receiving Trinitrobenzene Sulfonic Acid alone. Thus, all of the Th1-associated proinflammatory cytokines that were increased in Trinitrobenzene Sulfonic Acid colitis were increased to a significantly greater degree in mice receiving 1-mT in addition to Trinitrobenzene Sulfonic Acid. Inhibition of indoleamine 2,3-dioxygenase enhanced Th1 immune activation by increasing levels of Th1-related cytokines; this implied that indoleamine 2,3-dioxygenase functions to down-regulate Th1-mediated inflammation. The increased levels of IFN-γ seen in mice receiving Trinitrobenzene Sulfonic Acid plus 1 mT suggested a feedback loop in which IFN-γ up-regulated indoleamine 2,3-dioxygenase expression and indoleamine 2,3-dioxygenase expression decreased IFN-γ production through inhibition of T-cell activation and proliferation.

### EXAMPLE 6

This example illustrates the increase in indoleamine 2,3-dioxygenase following treatment with recombinant IFN-γ.

In order to obtain the various colonic lamina propria cellular subpopulations, lamina propria mononuclear cells were isolated from mouse colon and then fractionated using magnetic immunoselection. To isolate lamina propria mononuclear cells, mice were sacrificed, and their colons removed and placed in ice-cold PBS. Intestines were opened along the mesenteric attachment and isolation was performed as described in Newberry R D, J Immunol, 166:4465-4472, 2001. The isolated lamina propria mononuclear cells were then used for fractionation of lamina propria mononuclear cell subpopulations as described below or cultured in 96-well tissue culture plates at a density of 2.5×10⁶ cells/mL in RPMI 1640 medium (BioWhittaker, Walkersville, Md.) containing 2 mmol/L Glutamax 1 (L-Alanyl-L Glutamine; Life Technologies, Gaithersburg, Md.), 10 mmol/L HEPES, 1 mmol/L sodium pyruvate, 50 U/mL penicillin-50 mg/mL streptomycin, 50 μmol/Lβmercaptoethanol, and 10% FCS (HyClone, Logan, Utah) at 37° C. and 5% CO2 in the presence or absence of LPS 30 ng/mL (Sigma) and/or recombinant interferon γ (rIFN-γ) 30 ng/mL (R&D systems) for 24 hours at 37° C.

Fractionation of lamina propria mononuclear cells was performed using magnetic immunoselection. Colonic lamina propria mononuclear cells were resuspended at 2×10⁷ cells/mL in PBS with 1% bovine serum albumin (Fischer Scientific) and 1 mg/mL human IgG (Sandoz Pharmaceuticals, East Hanover, N.J.) for 20 minutes on ice. Cells were then incubated with biotin-conjugated anti-mouse B220 antibody (BD Biosciences) diluted in PBS containing 1% bovine serum albumin and 1 mg/mL human IgG for 20 minutes on ice, washed in PBS containing 1% BSA, incubated with streptavidin-conjugated microbeads (Miltenyi Biotech, Auburn, Calif.) per the manufacturer's directions, and magnetically sorted using MACS columns (Milteyni biotech). B220-depleted cells were then incubated with a biotin-conjugated anti-MHC II antibody (BD Biosciences, catalog No. 553540), which is cross-reactive with the H-2⁵ haplotype, in PBS containing 1% bovine serum albumin and 1 mg/mL human IgG for 20 minutes on ice. B220-depleted cells were washed in PBS containing 1% BSA and then incubated with streptavidin-conjugated microbeads and magnetically sorted as noted previously. Isolated cells were cultured in 96-well plates at a density of 5×10⁶ cells/mL in the presence or absence of rIFN-γ and/or LPS as described above. An aliquot of each isolated cell population was cultured overnight at 37° C. and 5% CO2 in the above media to allow for the phagocytosis of the streptavidin-coated microbeads and then analyzed by flow cytometry with the following antibodies/reagents: fluoresecin isothiocyanate (FITC)-conjugated anti-CD45, phycoerythrin (PE)-conjugated anti-CD19, FTC-conjugated anti-MHCII, biotin-conjugated anti-CD11c, biotin-conjugated anti-TCR-β, streptavidin-conjugated PE, appropriate isotype control antibodies (all available from BD Biosciences), and biotin-conjugated anti-F480 (Cedarlane Laboratories, Hornby, Ontario, Canada). Studies have shown that inhibition of indoleamine 2,3-dioxygenase activity in professional antigen presenting cells (macrophages and dendritic cells) augments T-cell-mediated inflammatory responses. Therefore, inhibition of indoleamine 2,3-dioxygenase activity in professional antigen presenting cells in the colon may account for the worsened histology, mortality, and increased production of the inflammatory cytokines that we observed. Professional antigen presenting cells account for approximately 10% of the lamina propria mononuclear cell population. To effectively enrich for professional antigen presenting cells, we first depleted B220+ cells (primarily B-lymphocytes) and then selected for MHC II⁺ cells (primarily professional antigen presenting cells). This MHCII⁺/B220⁻ fraction is 4-fold enriched (40% of total cells) for professional antigen presenting cells compared with unfractionated lamina propria mononuclear cells. Half of these antigen presenting cells are CD11c positive dendritic cells and half are F4/80 positive macrophages.

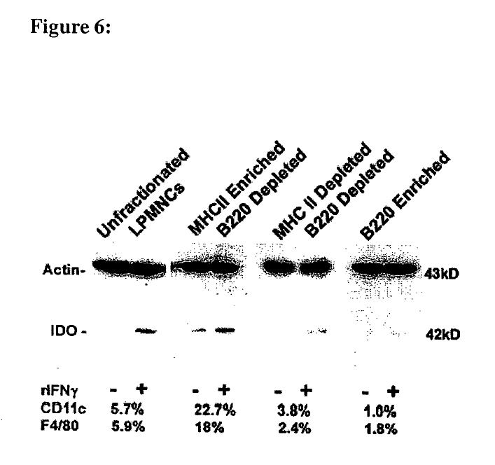

We determined whether indoleamine 2,3-dioxygenase is present in lamina propria mononuclear cell subpopulations isolated from colons of untreated mice and whether indoleamine 2,3-dioxygenase amounts increase when the cells are cultured with IFN-γ (FIG. 6). Indoleamine 2,3-dioxygenase was present in unstimulated lamina propria mononuclear cells; incubation with rIFN-γ resulted in a marked increase in indoleamine 2,3-dioxygenase expression. Indoleamine 2,3-dioxygenase was highly expressed at baseline in the MHCII⁺/B220⁻ lamina propria mononuclear cell population and was further induced in this population after incubation with rIFN-γ. Basal indoleamine 2,3-dioxygenase expression by this population was likely due to activation of these antigen presenting cells in situ because indoleamine 2,3-dioxygenase-expressing cells are seen in the colonic LP of unmanipulated animals (FIG. 2A). The B220⁺ enriched lamina propria mononuclear cell population did not express significant indoleamine 2,3-dioxygenase at baseline but did express indoleamine 2,3-dioxygenase after incubation with rIFN-γ. This population contained about 3% professional antigen presenting cells. The B220⁻/MHCII⁻ lamina propria mononuclear cell population expressed an intermediate amount of indoleamine 2,3-dioxygenase at baseline, which increased after incubation with rIFN-γ. This population included 6% professional antigen presenting cells, 22% T cells, and 67% CD45⁻ cells, which include stromal cells and endothelial cells.

These studies established that indoleamine 2,3-dioxygenase is highly present in lamina propria antigen presenting cells at baseline and is increased in amounts in multiple cell types after incubation with IFN-γ. In fractionated lamina propria mononuclear cells, indoleamine 2,3-dioxygenase expression at baseline was associated with B220⁻/MHC II⁺ cells, a cell fraction that is enriched for professional antigen presenting cells including (dendritic cells and macrophages). Although B220⁻/MHC II⁺ cells make up only 10% of the lamina propria mononuclear cell population, they account for the majority of the total baseline indoleamine 2,3-dioxygenase expression. Indoleamine 2,3-dioxygenase expression in this antigen presenting cell-rich population was induced further by incubation with rIFN-γ. Indoleamine 2,3-dioxygenase expression by antigen presenting cells has been described in a number of systems outside of the GI tract. In addition to antigen presenting cells, other cell types express indoleamine 2,3-dioxygenase under the influence of IFN-γ; these include epithelial and endothelial cells, fibroblasts, smooth muscle cells, and cells associated with the nervous system. Although the majority of rIFN-γ-inducible indoleamine 2,3-dioxygenase was expressed in the B220⁻/MHC II⁺ lamina propria mononuclear cell fraction, there was some indoleamine 2,3-dioxygenase expression after rIFN-γ incubation in both the B220⁻/MHC II⁻ and the B220⁺ lamina propria mononuclear cell fractions, suggesting the possibility of IFN-γ-induced indoleamine 2,3-dioxygenase expression in other cell types, including stromal cells and endothelial cells and the subset of dendritic cells that are B220⁺.

### EXAMPLE 7

This example illustrates decreased indoleamine 2,3-dioxygenase expression and increased inflammation of the colon of STAT-1 and IFN-γ knockout mice (STAT1⁻/⁻ and IFN-γReceptor −/−, respectively) in response to Trinitrobenzene Sulfonic Acid.

STAT1⁻/⁻ and IFN-γReceptor −/− mice were on the C57BL/6 background. C57BL/6 mice approximately 6 to 8 weeks of age and matched for age and gender with the knockouts were purchased from the Jackson Laboratory.

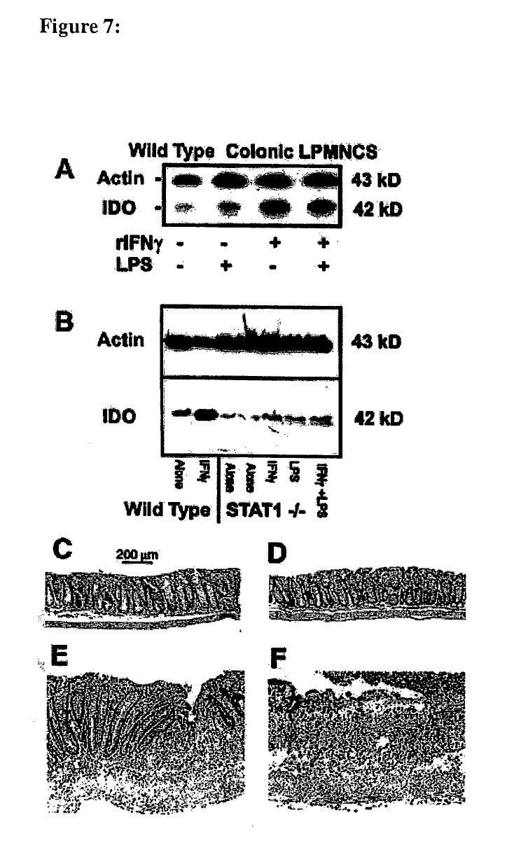

We determined whether STAT1⁻/⁻ mice had impaired lamina propria mononuclear cell indoleamine 2,3-dioxygenase induction in response to INF-γ and develop a more severe form of Trinitrobenzene Sulfonic Acid colitis as compared with wild-type controls. There was baseline indoleamine 2,3-dioxygenase expression in isolated colonic lamina propria mononuclear cells from C57BL/6 mice. This expression increased significantly following a 24-hour incubation with rIFN-γ. Culturing these cells with LPS for 24 hours produced a relatively weak indoleamine 2,3-dioxygenase induction compared with IFN-γ alone. IFN-γ alone induced indoleamine 2,3-dioxygenase to the same extent as the combination of LPS and IFN-γ (FIG. 7A). But, in STAT1⁻/⁻ mice, they demonstrated impaired lamina propria mononuclear cell indoleamine 2,3-dioxygenase induction in response to r IFN-γ and developed a more severe form of Trinitrobenzene Sulfonic Acid colitis as compared with wild-type controls. Colon lamina propria mononuclear cells from STAT1⁻/⁻ animals had a severely blunted indoleamine 2,3-dioxygenase protein induction in response to IFN-γ (FIG. 7B) but not necessarily LPS. When STAT1⁻/⁻ mice were given 2 mg Trinitrobenzene Sulfonic Acid, they developed significantly more inflammation and distal colonic injury compared with control C57BL/6 mice given the same dose of Trinitrobenzene Sulfonic Acid (FIG. 7C-F).

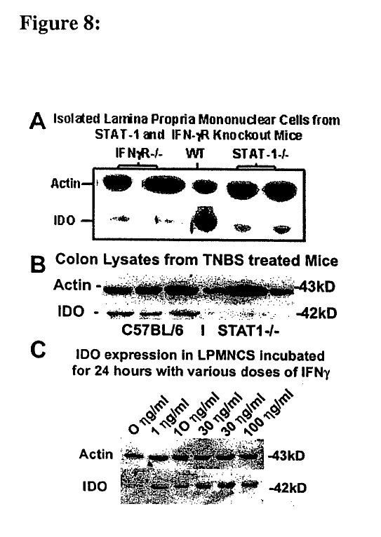

Furthermore, these studies showed that INF-γ induction of indoleamine 2,3-dioxygenase was blunted in lamina propria mononuclear cells isolated from STAT1⁻/⁻ mice, which lack a signaling molecule necessary for INF-γ responsiveness. IFN-γReceptor −/− mice, like the STAT-1 −/− mice, had decreased basal indoleamine 2,3-dioxygenase protein (FIG. 8A). Distal colon lysates from control C57BL/6 mice with Trinitrobenzene Sulfonic Acid colitis demonstrated increased indoleamine 2,3-dioxygenase protein content, while indoleamine 2,3-dioxygenase protein was barely detectable in distal colonic lysates from STAT-1 −/− animals in the presence of Trinitrobenzene Sulfonic Acid colitis (FIG. 8B). Extremely low concentration (less than 1 ng/ml) of recombinant IFN-γ was required to maximally increase indoleamine 2,3-dioxygenase protein in lamina propria mononuclear cells (FIG. 8C). This was evidence that INF-γ is essential for maximal indoleamine 2,3-dioxygenase expression because other known inducers (i.e. LPS) are not able to compensate for the INF-γ unresponsiveness in the STAT1⁻/⁻ animals. Trinitrobenzene Sulfonic Acid colitis was more severe in STAT1⁻/⁻ mice than in wild-type controls, suggesting a protective role for INF-γ in Trinitrobenzene Sulfonic Acid colitis via indoleamine 2,3-dioxygenase induction. Despite being considered a “proinflammatory” cytokine associated with Th1-mediated immune responses, there are several experimental colitis models in which INF-γ appears to function in an anti-inflammatory manner. INF-γ-deficient mice developed more severe crypt inflammation and colonic patch hypertrophy than do normal control animals in the setting of Trinitrobenzene Sulfonic Acid colitis. Also in the setting of Trinitrobenzene Sulfonic Acid colitis, colons from INF-γ receptor deficient mice contained increased numbers of macrophages and CD4+ T cells, and their caudal lymph nodes produced increased levels of proinflammatory cytokines. These published studies, along with our data from STAT1⁻/⁻ animals, suggested an increased inflammatory response to Trinitrobenzene Sulfonic Acid in the absence of INF-γ or its intracellular signaling pathway.

### EXAMPLE 8

This example illustrates that inhibiting indoleamine 2,3-dioxygenase in Trinitrobenzene Sulfonic Acid colitis increases lymphocyte proliferation.

Lymphocyte proliferation was determined by BrdU labeling of lamina propria lymphoid aggregates of mice treated with Trinitrobenzene Sulfonic Acid enemas plus subcutaneous pellets containing 1-methyl-tryptophan.

BrdU immunohistochemistry was performed to measure cellular proliferation in hypertrophied lymphoid aggregates within the lamina propria of Trinitrobenzene Sulfonic Acid±1-methyl-tryptophan treated mice. Mice were given rectal Trinitrobenzene Sulfonic Acid and subcutaneous pellets containing 1-methyl-tryptophan or placebo. Groups of mice were sacrificed at 4 days and 6 days after Trinitrobenzene Sulfonic Acid administration. Each mouse received BrdU two hours prior to sacrifice in order to label the S-phase cells. Four micro paraffin sections were prepared from the inflamed colon segments with macroscopically visible hypertrophied colonic patches then labeling was performed as previously described in Riehl T, Gasteroenterology, 118: 1106-16, 2000.

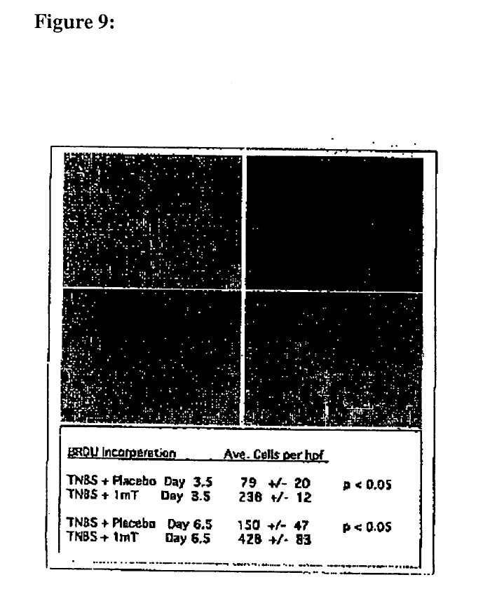

We determined that there were S phase lymphocytes in lymphoid aggregates from mice receiving Trinitrobenzene Sulfonic Acid plus placebo but significantly more S phase lymphocytes in lymphoid aggregates from mice receiving Trinitrobenzene Sulfonic Acid plus 1-methyl-tryptophan (FIG. 9). At each time-point shown, mice receiving Trinitrobenzene Sulfonic Acid plus 1-methyl-tryptophan demonstrated increased uptake of BrdU in mononuclear cells with a morphology consistent with lymphocytes. These studies showed that treatment with 1-methyl-tryptophan during Trinitrobenzene Sulfonic Acid colitis results in increased lymphocyte proliferation in colonic lymphoid aggregates. indoleamine 2,3-dioxygenase activity inhibits T cell proliferation and inhibition of indoleamine 2,3-dioxygenase with 1-methyl-tryptophan removes this block on T-cell proliferation. One consequence of this failure to down-regulate lymphocyte proliferation in the colon of 1-methyl-tryptophan treated animals was worsened colitis.

### EXAMPLE 9

This example illustrates the induction of indoleamine 2,3-dioxygenase by lipopolysaccharide (LPS) and cytotoxic T lymphocyte-associated antigen 4-immunoglobulin (CTLA-4-Ig) and the reduction of inflammation in colitis elicited by Trinitrobenzene Sulfonic Acid through systemic administration of LPS.

LPS (10 μg/mouse), an inducer of indoleamine 2,3-dioxygenase, was administered intraperitoneally to determine levels of indoleamine 2,3-dioxygenase and to determine the effect of LPS on cilitis.

To determine the effect of CTLA-4-Ig on indoleamine 2,3-dioxygenase, lamina propria mononuclear cells were cultured with various doses of CTLA-4-Ig, including 0, 10, 40, and 100 μg/ml.

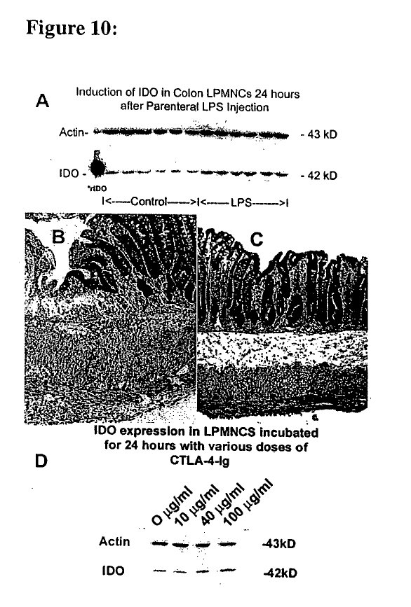

We determined whether systemic administration of LPS, an inducer of indoleamine 2,3-dioxygenase, in the days prior to Trinitrobenzene Sulfonic Acid administration, significantly reduced the inflammatory response to Trinitrobenzene Sulfonic Acid. Western blotting demonstrated a 3 to 4-fold increase in indoleamine 2,3-dioxygenase in lamina propria mononuclear cells isolated from SJL/J mice 24 hours after intraperitoneal administration of LPS (FIG. 10A). Furthermore, histological analysis demonstrated a markedly less inflammation in the LPS-treated mouse colon in response to Trinitrobenzene Sulfonic Acid (FIGS. 10B and C). Also, we demonstrated that indoleamine 2,3-dioxygenase is increased in lamina propria cells cultured with various doses of CTLA-4-Ig, the maximal increase being achieved at a CTLA-4-Ig dose of 40 μg/ml (FIG. 10D).

These studies established that increasing indoleamine 2,3-dioxygenase by administration of LPS prior to Trinitrobenzene Sulfonic Acid administration significantly diminished the resulting colitis. It is believed that CTLA-4-Ig administered systemically, will also decrease colitis produced tby Trinitrobenzene Sulfonic Acid.

All references cited in this specification are hereby incorporated by reference. Any discussion of references cited herein is intended merely to summarize the assertions made by their authors and no admission is made that any reference or portion thereof constitutes relevant prior art. Applicants reserve the right to challenge the accuracy and pertinency of the cited references.

The description of the invention is merely exemplary in nature and, thus, variations that do not depart from the gist of the invention are intended to be within the scope of the invention. Such variations are not to be regarded as a departure from the spirit and scope of the invention.

TABLE 1
Table 1. Morphologic and Histologic Scores
for Mice Treated with TNBS Enemas Plus Subcutaneous
Pellets Containing Placebo or 1-mT

| | | P value |
|---|---|---|
| | Histologic scores | |
| Day 3 | | |
| TNBS + placebo | 5.3 ± 0.6 | 0.51 |
| TNBS + 1-mT | 5.7 ± 0.6 | |
| Day 4 | | |
| TNBS + placebo | 5.0 ± 1.1 | 0.004* |
| TNBS + 1-mT | 6.7 ± 0.5 | |
| Day 6 | | |
| TNBS + placebo | 4.0 ± 1.0 | 0.002* |
| TNBS + 1-mT | 7.0 ± 0.0 | |
| | Morphologic scores | |
| Day 4 | | |
| TNBS + placebo | 2.7 ± 0.9 | 0.008* |
| TNBS + 1-mT | 4.3 ± 1.3 | |

1. A method for treating a patient having inflammatory bowel disease, the method comprising administering to the patient an anti-inflammatory amount of an inducer of 2,3-dioxygenase in antigen presenting cells of the patient's gastrointestinal tract.

2. A method of claim 1, wherein the inducer increases expression of indoleamine 2,3-dioxygenase in antigen presenting cells of the patient's gastrointestinal tract.

3. A method of claim 1, wherein the inducer comprises a bacterial lipopolysaccharide, an interferon-γ or a cytotoxic T lymphocyte-associated antigen 4.

4. A method of claim 3, wherein the bacterial lipopolysaccharide is an E. coli lipopolysaccharide.

5. A method of claim 3, wherein the interferon-γ is an interferon-γ-Ig fusion polypeptide or a pegylated interferon-γ.

6. A method of claim 3, wherein the cytotoxic T lymphocyte-associated protein 4 is a cytotoxic T lymphocyte-associated protein 4-Ig fusion polypeptide or a pegylated cytotoxic T lymphocyte-associated protein 4-Ig.

7. A method of claim 1, wherein the antigen presenting cells are professional antigen presenting cells.

8. A method of claim 7, wherein the antigen presenting cells are lamina propria mononuclear cells.

9. A method of claim 1, wherein the antigen presenting cells are macrophages or dendritic cells.

10. A method of claim 1, wherein the patient is a human patient.

11. A method of claim 1, wherein the inflammatory bowel disease is ulcerative colitis.

12. A method of claim 1, wherein the inflammatory bowel disease is Crohn's disease.

13. A method of claim 1, wherein the administering comprises administering systemically.

14. A method of claim 13, wherein the administering comprises administer systemically by intravenous infusion.

15. A method of claim 1, further comprising administration of a substance selected from the group consisting of 5-aminosalicylates, corticosteroids and azathioprine

16. A method of downregulating a T helper 1 cell proliferation response in inflammation within the gastrointestinal tract in a mammalian subject having inflammatory bowel disease, the method comprising administering to the subject a pharmaceutical composition comprising an inducer of indoleamine 2,3-dioxygenase in antigen presenting cells of the subject's gastrointestinal tract.

17. A method of claim 16, wherein the inducer increases expression of indoleamine 2,3-dioxygenase in antigen presenting cells of the patient's gastrointestinal tract.

18. A method of claim 16, wherein the inducer comprises a bacterial lipopolysaccharide, an interferon-γ or a cytotoxic T lymphocyte-associated antigen 4.

19. A method of claim 18, wherein the bacterial lipopolysaccharide is an E. coli lipopolysaccharide.

20. A method of claim 18, wherein the interferon-γ is an interferon-γ-Ig fusion polypeptide or a pegylated interferon-γ.

21. A method of claim 18, wherein the cytotoxic T lymphocyte-associated protein 4 is a cytotoxic T lymphocyte-associated protein 4-Ig fusion polypeptide or a pegylated cytotoxic T lymphocyte-associated protein 4-Ig.

22. A method of claim 16, wherein the antigen presenting cells are professional antigen presenting cells.

23. A method of claim 16, wherein the antigen presenting cells are lamina propria mononuclear cells.

24. A method of claim 16, wherein the antigen presenting cells are macrophages or dendritic cells.

25. A method of claim 16, wherein the mammalian subject is a human.

26. A method of claim 16, wherein the inflammatory bowel disease is ulcerative colitis.

27. A method of claim 16, wherein the inflammatory bowel disease is Crohn's disease.

28. A method of claim 16, wherein the administrating comprises administrating systemically.

29. A method of claim 28, wherein the administrating systemically comprises administrating systemically by infusion.

30. A method of claim 16, further comprising administration of a substance selected from the group consisting of 5-aminosalicylates, corticosteroids and azathioprine

31. A packaged pharmaceutical comprising an anti-inflammatory amount of an inducer of indoleamine 2,3-dioxygenase in antigen presenting cells of a patient's gastrointestinal tract, in a pharmaceutically acceptable formulation and instructions for using said substance for treating inflammatory bowel disease in a patient.

32. A packaged pharmaceutical of claim 31, wherein the inducer increases expression of indoleamine 2,3-dioxygenase in antigen presenting cells of the patient's gastrointestinal tract.

33. A packaged pharmaceutical of claim 31, wherein the inducer comprises a bacterial lipopolysaccharide, an interferon-γ or a cytotoxic T lymphocyte-associated antigen 4.

34. A method of claim 33, wherein the bacterial lipopolysaccharide is an E. coli lipopolysaccharide.

35. A packaged pharmaceutical of claim 33, wherein the interferon-γ is an interferon-γ-Ig fusion polypeptide or a pegylated interferon-γ.

36. A packaged pharmaceutical of claim 33, wherein the cytotoxic T lymphocyte-associated protein 4 is a cytotoxic T lymphocyte-associated protein 4-Ig fusion polypeptide or a pegylated cytotoxic T lymphocyte-associated protein 4-Ig.

37. A packaged pharmaceutical of claim 31, wherein the antigen presenting cells are professional antigen presenting cells.

38. A packaged pharmaceutical of claim 37, wherein the antigen presenting cells are lamina propria mononuclear cells.

39. A packaged pharmaceutical of claim 31, wherein the antigen presenting cells are macrophages or dendritic cells.

40. A packaged pharmaceutical of claim 31, wherein the patient is a human patient.

41. A packaged pharmaceutical of claim 31, wherein the inflammatory bowel disease is ulcerative colitis.

42. A packaged pharmaceutical of claim 31, wherein the inflammatory bowel disease is Crohn's disease.

43. A packaged pharmaceutical of claim 31, wherein the inducer is in a formulation suitable for intraperitoneal infusion.

44. A packaged pharmaceutical of claim 31, wherein the inducer is in a formulation suitable for systemic administration.

45. A packaged pharmaceutical of claim 43, wherein the inducer is in a formulation suitable for intravenous infusion.

46. A packaged pharmaceutical of claim 31 further comprising a substance selected from the group consisting of 5-aminosalicylates, corticosteroids and azathioprine, in a pharmaceutically acceptable formulation.
